(19) United States
(12) Patent Application Publication
(10) Pub. No.: US 2007/0244056 A1
(43) Pub. Date: Oct. 18, 2007

(54) Combination Therapy With Glatiramer Acetate and Riluzole

(76) Inventors: Liat Hayardeny, Tel- Aviv (IL); Ety Klinger, Tel-Aviv (IL); Eran Blaugrund, Rehovot (IL)

Correspondence Address: COOPER & DUNHAM, LLP, 1185 AVENUE OF THE AMERICAS, NEW YORK, NY, 10036, US

(21) Appl. No.: 10/591,195

(22) PCT Filed: Mar. 3, 2005
(86) PCT No.: PCT/US05/07072

§ 371 Date: Sep. 29, 2006

Related U.S. Application Data
(60) Provisional application No. 60/549,975, filed on Mar. 3, 2004.
(60) Provisional application No. 60/549,976, filed on Mar. 3, 2004.
(60) Provisional application No. 60/549,974, filed on Mar. 3, 2004.

(51) Int. Cl. A61K 38/03; A61K 38/07; A61P 25/00

(52) U.S. Cl. 514/18

(57) ABSTRACT

The subject invention provides a method of providing neuroprotection to the central or peripheral nervous system of a subject in need of such neuroprotection comprising periodically administering to the subject an amount of glatiramer acetate and an amount of 2-amino-6-trifluoromethoxybenzathiazole, wherein the amounts when taken together are effective to provide neuroprotection to the central or peripheral nervous system of the subject. The subject invention also provides a package comprising glatiramer acetate, 2-amino-6-trifluorormethoxybenzothiazole and instructions for use together to provide neuroprotection to the central or peripheral nervous system of a subject in need of such neuroprotection. Additionally, the subject invention provides a pharmaceutical composition comprising an amount of glatiramer acetate and an amount of 2-amino-6-trifluorormethoxybenzothiazole, wherein the amounts when taken together are effective to provide neuroprotection to the central or peripheral nervous system of the subject. The subject invention further provides a pharmaceutical combination comprising separate dosage forms of an amount of glatiramer acetate and an amount of 2-amino-6-trifluorormethoxybenzothiazole, which combination is useful to provide neuroprotection to the central or peripheral nervous system of the subject. In addition, the combination therapy may be used to treat a subject afflicted with multiple sclerosis or one afflicted with amyotrophic lateral sclerosis.

Throughout this application, various publications are referenced in parenthesis. Full citations for these publications may be found listed in alphabetical order at the end of the specification immediately preceding the claims. The disclosures of these publications in their entireties are hereby incorporated by reference into this application in order to more fully describe the state of the art to which this invention pertains.

### FIELD OF THE INVENTION

The subject invention relates to combination therapy using glatiramer acetate and riluzole for neuroprotection, multiple sclerosis, and amyotrophic lateral sclerosis.

### BACKGROUND OF THE INVENTION

Neuroprotection refers to protection of the central or peripheral nervous system from neuronal loss, axonal loss and/or myelin loss. Providing neuroprotection is one way to effect the treatment of neurodegenerative conditions and neurotrauma.

One of the more common neurologic diseases in human adults is multiple sclerosis. This condition is a chronic, inflammatory CNS disease characterized pathologically by demyelination. There are five main forms of multiple sclerosis: 1) benign multiple sclerosis; 2) relapsing-remitting multiple sclerosis (RR-MS); 3) secondary progressive multiple sclerosis (SP-MS); 4) primary progressive multiple sclerosis (PP-MS); and 5) progressive-relapsing multiple sclerosis (PR-MS). Benign multiple sclerosis is characterized by 1-2 exacerbations with complete recovery, no lasting disability and no disease progression for 10-15 years after the initial onset. Benign multiple sclerosis may, however, progress into other forms of multiple sclerosis. Patients suffering from RR-MS experience sporadic exacerbations or relapses, as well as periods of remission. Lesions and evidence of axonal loss may or may not be visible on MRI for patients with RR-MS. SP-MS may evolve from RR-MS. Patients afflicted with SP-MS have relapses, a diminishing degree of recovery during remissions, less frequent remissions and more pronounced neurological deficits than RR-MS patients. Enlarged ventricles, which are markers for atrophy of the corpus callosum, midline center and spinal cord, are visible on MRI of patients with SP-MS. PP-MS is characterized by a steady progression of increasing neurological deficits without distinct attacks or remissions. Cerebral lesions, diffuse spinal cord damage and evidence of axonal loss are evident on the MRI of patients with PP-MS. PR-MS has periods of acute exacerbations while proceeding along a course of increasing neurological deficits without remissions. Lesions are evident on MRI of patients suffering from PR-MS (Multiple sclerosis: its diagnosis, symptoms, types and stages).

Researchers have hypothesized that multiple sclerosis is an autoimmune disease (Compston; Hafler and Weiner; Olsson). An autoimmune hypothesis is supported by the experimental allergic encephalomyelitis (EAE) model of multiple sclerosis, where the injection of certain myelin components into genetically susceptible animals leads to T cell-mediated CNS demyelination (Parkman). Another theory regarding the pathogenesis of multiple sclerosis is that a virus, bacteria or other agent, precipitates an inflammatory response in the CNS, which leads to either direct or indirect (“bystander”) myelin destruction, potentially with an induced autoimmune component (Lampert; Martyn). Another experimental model of multiple sclerosis, Theiler's murine encephalomyelitis virus (TMEV)(Dal Canto and Lipton; Rodriguez et al.), supports the theory that a foreign agent initiates multiple sclerosis. In the TMEV model, injection of the virus results in spinal cord demyelination.

Amyotrophic lateral sclerosis (ALS), also known as Lou Gehrig's disease, is a neurodegenerative disease that occurs when motor neurons degenerate, causing the muscles under their control to atrophy. Symptoms may include loss of motor control in one's extremities, twitching, cramping and difficulties in speaking, swallowing and breathing. Death usually occurs within 5 years of diagnosis (Amyotrophic Lateral Sclerosis Information Page, National Institute of Neurological Disorders and Stroke). The etiology and pathogenesis of ALS are not known, although a number of hypotheses have been advanced (Physician's Desk Reference, 2002). One hypothesis is that motor neurons, made vulnerable through either genetic predisposition or environmental factors, are injured by glutamate. There is evidence that mitochondrial damage and oxidative stress plays a role in human sporadic ALS (Ludolph A. C. et al.; Vielhaber S. et al.). In some cases of familial ALS, the enzyme superoxide dismutase has been found to be defective (Physician's Desk Reference, 2002).

Glatiramer acetate, also known as Copolymer 1 has been shown to be effective in treating multiple sclerosis (MS) (Lampert, P. W.). Daily subcutaneous injections of glatiramer acetate (20 mg/injection) reduce relapse rates, progression of disability, appearance of new lesions by magnetic resonance imaging (MRI), (Johnson, K. P. et al.) and appearance of “black holes” (Filippi, M. et al.).

COPAXONE® is the brand name for a formulation containing glatiramer acetate as the active ingredient. Glatiramer acetate is approved for reducing the frequency of relapses in relapsing-remitting multiple sclerosis. Glatiramer acetate consists of the acetate salts of synthetic polypeptides containing four naturally occurring amino acids: L-glutamic acid, L-alanine, L-tyrosine, and L-lysine with an average molar fraction in COPAXONE® of 0.141, 0.427, 0.095 and 0.338, respectively. In COPAXONE®, the average molecular weight of the glatiramer acetate is 4,700-11,000 daltons. Chemically, glatiramer acetate is designated L-glutamic acid polymer with L-alanine, L-lysine and L-tyrosine, acetate (salt). Its structural formula is:
(Glu, Ala, Lys, Tyr)ₓCH₃COOH (C₅H₉NO₄.C₃H₇NO₂.C₆H₁₄N₂O₂.C₉H₁₁NO₃)ₓ.XC₂H₄O₂
CAS—147245-92-9.

The recommended dosing schedule of COPAXONE® for relapsing-remitting multiple sclerosis is 20 mg per day injected subcutaneously (Physician's Desk Reference, 2003; see also U.S. Pat. Nos. 3,849,550; 5,800,808; 5,858,964, 5,981,589; 6,048,898; 6,054,430; 6,214,791; 6,342,476; and 6,362,161, all of which are hereby incorporated by reference).

Riluzole is a member of the benzothiazole class. Chemically, riluzole is 2-amino-6-trifluoromethoxy benzothiazole. Its molecular formula is C₈H₅F₃N₂OS and its molecular weight is 234.2 (Physician's Desk Reference, 2002).

RILUTEK® is a commercially available formulation of riluzole (2-amino-6-trifluoromethoxy benzothiazole), which is indicated for the treatment of patients with amyotrophic lateral sclerosis (ALS). RILUTEK® extends survival and/or time to tracheostomy. The recommended dose for RILUTEK® is 50 mg every 12 hours. RILUTEK® should be administered at least one hour before or at least two hours after a meal (Physician's Desk Reference, 2003). PCT Publication No. WO 01/95907 disclosed the results of testing 2-amino-6-trifluoromethoxy benzothiazole in an experimental autoimmune [sic] [allergic] encephalomyelitis (EAE), a murine model of multiple sclerosis. The PCT Publication suggested that 2-amino-6-trifluoromethoxy benzothiazole might be useful for the treatment of multiple sclerosis, but did not test whether 2-amino-6-trifluoromethoxy benzothiazole is effective to alleviate symptoms of any specific form of multiple sclerosis in humans.

PCT Publication No. WO 00/74676 disclosed a study in which 2-amino-6-trifluoromethoxy benzothiazole alone, was administered to human patients afflicted with an unspecified form of multiple sclerosis. This PCT publication suggested that 2-amino-6-trifluoromethoxy benzothiazole may be used to treat all forms of multiple sclerosis, and also suggests that 2-amino-6-trifluoromethoxy benzothiazole may be combined, with other agents useful in treating multiple sclerosis, such as interferons (especially type I interferons), steroids, pain relievers, muscle relaxants, copaxone [sic] [COPAXONE®], immunosuppressants or anti-depressants. However, the experimental data in this PCT Publication showed the increase in T2 lesion load in the brain did not alter much under treatment, but the accumulation of hypointense lesions showed a trend toward reduction. No effect on EDSS score was seen. Such data is not supportive of the suggestion made and certainly doesn't suggest reducing frequency of relapses in relapsing-remitting multiple sclerosis patients.

PCT International Publication Nos. WO 01/52878 and WO 01/93893 list ALS as one of a number of indications which may possibly be affected by glatiramer acetate, but do not test glatiramer acetate for the treatment of ALS.

The administration of two drugs to treat a given condition, such as a form of multiple sclerosis, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry. In vivo drug metabolism/drug interaction studies—study design, data analysis, and recommendations for dosing and labeling). Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry. In vivo drug metabolism/drug interaction studies—study design, data analysis, and recommendations for dosing and labeling). The interaction may also heighten or lessen the side effects of each drug. Hence, upon administration of two drugs to treat a disease, it is unpredictable what change will occur in the negative side profile of each drug.

Additionally, it is accurately difficult to predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry. In vivo drug metabolism/drug interaction studies—study design, data analysis, and recommendations for dosing and labeling).

Thus, the success of one drug or each drug alone in an in vitro model, an animal model, or in humans, may not correlate into efficacy when both drugs are administered to humans.

In accordance with the subject invention, glatiramer acetate and 2-amino-6-trifluoromethoxybenzathiazole are effective in combination to provide neuroprotection in various neurodegenerative diseases, including multiple sclerosis (MS) (particularly, relapsing-remitting multiple sclerosis), amyotrophic lateral sclerosis (ALS), acute disseminated encephalomyelitis, adrenleukodystrophy, adreno-myeloneuropathy, Leber's hereditary optic atrophy, Human Lymphotrophic T-cell Virus I (HTLVI)-associated myelopathy, acute viral encephalitis, aseptic meningitis, virus-induced demyelination, demyelinating genetic diseases, transverse myelitis, Progressive Multifocal Leucoencephalopathy, a nutritional metabolic disorder, acute glaucoma, chronic glaucoma, close-angle glaucoma, open-angle glaucoma, optic neuritis and systemic lupus erythematosus. The use of glatiramer acetate and riluzole in combination according to the subject invention also provides neuroprotection in various neurotrauma resulting from a traumatic event, such as head trauma, spinal trauma, neurotoxic injury, eye injury, stroke, ischemia, hypoxia, and anoxia. Additionally according to the subject invention, the use of glatiramer acetate and riluzole in combination provides neuroprotection including protection against toxic levels of glutamate or toxic levels of monoamine oxidase-B activity.

### SUMMARY OF THE INVENTION

The subject invention provides a method of providing neuroprotection to the central or peripheral nervous system of a subject in need of such neuroprotection comprising periodically administering to the subject an amount of glatiramer acetate and an amount of 2-amino-6-trifluoromethoxybenzathiazole, wherein the amounts when taken together are effective to provide neuroprotection to the central or peripheral nervous system of the subject.

The subject invention further provides a method of treating a subject afflicted with a form of multiple sclerosis comprising periodically administering to the subject an amount of glatiramer acetate and an amount of 2-amino-6-trifluoromethoxybenzathiazole, wherein the amounts when taken together are effective to alleviate a symptom of the form of multiple sclerosis in the subject so as to thereby treat the subject.

The subject invention still further provides a method of treating a subject afflicted with amyotrophic lateral sclerosis comprising periodically administering to the subject an amount of glatiramer acetate and an amount of 2-amino-6-trifluoromethoxybenzathiazole, wherein the amounts when taken together are effective to alleviate a symptom of amyotrophic lateral sclerosis in the subject so as to thereby treat the subject.

In addition, the subject invention provides a package comprising
i) a first pharmaceutical composition comprising an amount of glatiramer acetate and a pharmaceutically acceptable carrier;
ii) a second pharmaceutical composition comprising an amount of 2-amino-6-trifluorormethoxybenzothiazole and a pharmaceutically acceptable carrier; and
iii) instructions for use of the first and second pharmaceutical compositions together to alleviate a symptom of multiple sclerosis or a symptom of amyotrophic lateral sclerosis in, or to provide neuroprotection to, a subject.

The subject invention further provides a pharmaceutical composition comprising an amount of glatiramer acetate and an amount of 2-amino-6-trifluorormethoxybenzothiazole, wherein the amounts when taken together are effective to alleviate a symptom of amyotrophic lateral sclerosis in a subject, or are effective to alleviate a symptom of multiple sclerosis, or and effective to provide neuroprotection to the subject.

### DETAILED DESCRIPTION OF THE INVENTION

The subject invention provides a method of providing neuroprotection to the central or peripheral nervous system of a subject, e.g. a human being in need of such neuroprotection comprising periodically administering to the subject an amount of glatiramer acetate and an amount of 2-amino-6-trifluoromethoxybenzathiazole, wherein the amounts when taken together are effective to provide neuroprotection to the central or peripheral nervous system of the subject.

In one embodiment, providing neuroprotection comprises treating a neurodegenerative disease.

In various embodiments, the neurodegenerative disease is alternatively multiple sclerosis, amyotrophic lateral sclerosis, acute disseminated encephalomyelitis, transverse myelitis, demyelinating genetic diseases, virus-induced demyelination, Progressive Multifocal Leucoencephalopathy, Human Lymphotrophic T-cell Virus I (HTLVI)-associated myelopathy, a nutritional metabolic disorder, acute glaucoma, chronic glaucoma, close-angle glaucoma, open-angle glaucoma, optic neuritis or systemic lupus erythematosus.

In a specific embodiment, the neurodegenerative disease is multiple sclerosis.

In another specific embodiment, the neurodegenerative disease is amyotrophic lateral sclerosis.

In yet another embodiment, the neurodegenerative disease is acute close-angle glaucoma.

In still another embodiment, the neurodegenerative disease is optic neuritis.

In a further embodiment, the neurodegenerative disease is systemic lupus erythematosus.

In an additional embodiment, the neurodegenerative disease is a nutritional metabolic disorder, e.g. a vitamin deficiency or central pontine myelinolysis. The vitamin deficiency may be vitamin B₁₂ deficiency.

In an additional embodiment, providing neuroprotection comprises treating neurotrauma, e.g. neurotrauma which is a result of a traumatic event selected from the group consisting of head trauma, spinal trauma, neurotoxic injury, eye injury, stroke, ischemia, hypoxia, and anoxia.

In one embodiment, providing neuroprotection comprises providing protection against toxic levels of glutamate.

In an added embodiment, the neuroprotection comprises protection against toxic levels of monoamine oxidase-B activity.

In a further embodiment, each of the amount of glatiramer acetate when taken alone, and the amount of 2-amino-6-trifluoromethoxybenzathiazole when taken alone is effective to provide neuroprotection.

In some embodiments, either the amount of glatiramer acetate when taken alone, the amount of 2-amino-6-trifluoromethoxybenzathiazole when taken alone or each such amount when taken alone is not effective to provide neuroprotection.

In certain embodiments, the amount of glatiramer acetate may be 10 to 80 mg; or 12 to 70 mg; or 14 to 60 mg; or 16 to 50 mg; or 18 to 40 mg; or 20 to 30 mg; or 20 mg. For each amount of glatiramer acetate, the amount of 2-amino-6-trifluoromethoxybenzathiazole may be 25 to 75 mg; or 35 to 65 mg; or 45 to 55 mg; or 50 mg.

Alternatively, the amount of glatiramer acetate may be in the range from 10 to 600 mg/week; or 100 to 550 mg/week; or 150 to 500 mg/week; or 200 to 450 mg/week; or 250 to 400 mg/week; or 300 to 350 mg/week; or 300 mg/week.

In another embodiment, the amount of glatiramer acetate may be in the range from 50 to 150 mg/day; or 60 to 140 mg/day; or 70 to 130 mg/day; or 80 to 120 mg/day; or 90 to 110 mg/day; or 100 mg/day.

Alternatively, the amount of glatiramer acetate may be in the range from 10 to 80 mg/day; or 12 to 70 mg/day; or 14 to 60 mg/day; or 16 to 50 mg/day; or 18 to 40 mg/day; or 19 to 30 mg/day; or 20 mg/day.

In certain embodiments, the periodic administration of glatiramer acetate is effected daily; twice daily at one half the amount; or once every 3 to 11 days; or once every 5 to 9 days; or once every 7 days; or once every 24 hours.

For each administration schedule of glatiramer acetate, the 2-amino-6-trifluoromethoxybenzathiazole may be administered once every 8 to 16 hours; or once every 10 to 14 hours; or once every 12 hours.

In an embodiment, the periodic administration of 2-amino-6-trifluoromethoxybenzathiazole is effected at least one hours before or at least two hours after a meal.

In a further embodiment, the administration of the glatiramer acetate substantially precedes the administration of the 2-amino-6-trifluoromethoxybenzathiazole.

In an added embodiment, the administration of the 2-amino-6-trifluoromethoxybenzathiazole substantially precedes the administration of the glatiramer acetate.

In one embodiment, the glatiramer acetate and the 2-amino-6-trifluoromethoxybenzathiazole may be administered for a period of time of at least 4 days. In a further embodiment, the period of time may be 5 days to 5 years; or 10 days to 3 years; or 2 weeks to 1 year; or 1 month to 6 months; or 3 months to 4 months. In yet another embodiment, the glatiramer acetate and the 2-amino-6-trifluoromethoxybenzathiazole may be administered for the lifetime of the subject.

The administration of 2-amino-6-trifluoromethoxybenzathiazole or glatiramer acetate may each independently be oral, nasal, pulmonary, parenteral, intravenous, intra-articular, transdermal, intradermal, subcutaneous, topical, intramuscular, rectal, intrathecal, intraocular, buccal or by gavage. For 2-amino-6-trifluoromethoxybenzathiazole, the preferred route of administration is oral or by gavage. The preferred route of administration for glatiramer acetate is subcutaneous or oral. One of skill in the art would recognize that doses at the higher end of the range may be required for oral administration.

In one embodiment, the administration of the glatiramer acetate may be subcutaneous, intraperitoneal, intravenous, intramuscular, intraocular or oral and the administration of the 2-amino-6-trifluoromethoxybenzathiazole may be oral. In another embodiment, the administration of the glatiramer acetate may be subcutaneous and the administration of the 2-amino-6-trifluoromethoxybenzathiazole may be oral.

The subject invention also provides a package comprising
i) a first pharmaceutical composition comprising an amount of glatiramer acetate and a pharmaceutically acceptable carrier;
ii) a second pharmaceutical composition comprising an amount of 2-amino-6-trifluorormethoxybenzothiazole and a pharmaceutically acceptable carrier; and
iii) instructions for use of the first and second pharmaceutical compositions together to provide neuroprotection to a subject.

In an embodiment of the package, the amount of glatiramer acetate may be in the range from 10 to 600 mg; or 100 to 550 mg; or 150 to 500 mg; or 200 to 450 mg; or 250 to 400 mg; or 300 to 350 mg; or 300 mg.

In another embodiment of the package, the amount of glatiramer acetate may be in the range from 10 to 80 mg; or 12 to 70 mg; or 14 to 60 mg; or 16 to 50 mg; or 18 to 40 mg; or 19 to 30 mg; or 20 mg.

Alternatively, the amount of glatiramer acetate in the package may be in the range from 50 to 150 mg; or 60 to 140 mg; or 70 to 130 mg; or 80 to 120 mg; or 90 to 110 mg; or 100 mg.

For each amount of glatiramer acetate in the package, the amount of 2-amino-6-trifluoromethoxybenzathiazole in the package may be 25-75 mg; or 35-65 mg; or 45-55 mg; or 50 mg.

In one embodiment of the package, providing neuroprotection comprises treating a neurodegenerative disease.

In another embodiment of the package, the neurodegenerative disease is multiple sclerosis, amyotrophic lateral sclerosis, acute disseminated encephalomyelitis, transverse myelitis, demyelinating genetic diseases, virus-induced demyelination, Progressive Multifocal Leucoencephalopathy, Human Lymphotrophic T-cell Virus I (HTLVI)-associated myelopathy, a nutritional metabolic disorder, acute glaucoma, chronic glaucoma, close-angle glaucoma, open-angle glaucoma optic neuritis or systemic lupus erythematosus.

In a further embodiment of the package, the nutritional metabolic disorder is vitamin deficiency or central pontine myelinolysis.

In yet another embodiment of the package, the vitamin deficiency is vitamin B₁₂ deficiency.

In an additional embodiment of the package, the neuroprotection comprises treatment of neurotrauma.

In one embodiment of the package, the neurotrauma is a result of a traumatic event selected from the group consisting of head trauma, spinal trauma, neurotoxic injury, eye injury, stroke, ischemia, hypoxia, and anoxia.

In a further embodiment of the package, the neuroprotection comprises protection against toxic levels of glutamate.

In still another embodiment of the package, the neuroprotection comprises protection against toxic levels of monoamine oxidase-B activity.

The subject invention further provides a pharmaceutical composition comprising an amount of glatiramer acetate and an amount of 2-amino-6-trifluorormethoxybenzothiazole, wherein the amounts when taken together are effective to provide neuroprotection to a subject.

In one embodiment of the pharmaceutical composition, each of the amount of glatiramer acetate when taken alone and the amount of 2-amino-6-trifluoromethoxybenzathiazole when taken alone is effective to provide neuroprotection.

In another embodiment of the pharmaceutical composition, either of the amount of glatiramer acetate when taken alone, or the amount of 2-amino-6-trifluoromethoxybenzathiazole when taken alone or each such amount when taken alone is not effective to provide neuroprotection.

In an embodiment of the pharmaceutical composition, providing neuroprotection comprises treating a neurodegenerative disease.

In a further embodiment of the pharmaceutical composition, the neurodegenerative disease is multiple sclerosis, amyotrophic lateral sclerosis, acute disseminated encephalomyelitis, transverse myelitis, demyelinating genetic diseases, virus-induced demyelination, Progressive Multifocal Leucoencephalopathy, Human Lymphotrophic T-cell Virus I (HTLVI)-associated myelopathy, a nutritional metabolic disorder, acute glaucoma, chronic glaucoma, close-angle glaucoma, open-angle glaucoma optic neuritis or systemic lupus erythematosus.

In another embodiment of the pharmaceutical composition, the nutritional metabolic disorder is vitamin deficiency or central pontine myelinolysis.

In an added embodiment of the pharmaceutical composition, the vitamin deficiency is vitamin B₁₂ deficiency.

In one embodiment of the pharmaceutical composition, the neuroprotection comprises treatment of neurotrauma.

In still another embodiment of the pharmaceutical composition, the neurotrauma is a result of a traumatic event selected from the group consisting of head trauma, spinal trauma, neurotoxic injury, eye injury, stroke, ischemia, hypoxia, and anoxia.

In an additional embodiment of the pharmaceutical composition, the neuroprotection comprises protection against toxic levels of glutamate.

In a further embodiment of the pharmaceutical composition, the neuroprotection comprises protection against toxic levels of monoamine oxidase-B activity.

The subject invention further provides a pharmaceutical combination comprising separate dosage forms of an amount of glatiramer acetate and an amount of 2-amino-6-trifluorormethoxybenzothiazole, which combination is useful to provide neuroprotection to a subject.

In an embodiment of the pharmaceutical combination, each of the amount of glatiramer acetate when taken alone and the amount of 2-amino-6-trifluoromethoxybenzathiazole when taken alone is provide neuroprotection.

In an additional embodiment of the pharmaceutical combination, either of the amount of glatiramer acetate when taken alone, the amount of 2-amino-6-trifluoromethoxybenzathiazole when taken alone or each such amount when taken alone is not effective to provide neuroprotection.

In a further embodiment, the pharmaceutical combination may be for simultaneous, separate or sequential use to provide neuroprotection to the subject.

In an embodiment of the pharmaceutical combination, providing neuroprotection comprises treating a neurodegenerative disease.

In still another embodiment of the pharmaceutical combination, the neurodegenerative disease is multiple sclerosis, amyotrophic lateral sclerosis, acute disseminated encephalomyelitis, transverse myelitis, demyelinating genetic diseases, virus-induced demyelination, Progressive Multifocal Leucoencephalopathy, Human Lymphotrophic T-cell Virus I (HTLVI)-associated myelopathy, a nutritional metabolic disorder, acute glaucoma, chronic glaucoma, close-angle glaucoma, open-angle glaucoma optic neuritis or systemic lupus erythematosus.

In an embodiment of the pharmaceutical combination, the nutritional metabolic disorder is vitamin deficiency or central pontine myelinolysis.

In yet another embodiment of the pharmaceutical combination, the vitamin deficiency is vitamin B₁₂ deficiency.

In one embodiment of the pharmaceutical combination, the neuroprotection comprises treatment of neurotrauma.

In another embodiment of the pharmaceutical combination, the neurotrauma is a result of a traumatic event selected from the group consisting of head trauma, spinal trauma, neurotoxic injury, eye injury, stroke, ischemia, hypoxia, and anoxia.

In a further embodiment of the pharmaceutical combination, the neuroprotection comprises protection against toxic levels of glutamate.

In still another embodiment of the pharmaceutical combination, the neuroprotection comprises protection against toxic levels of monoamine oxidase-B activity.

Providing neuroprotection includes protecting the central or peripheral nervous system from neurotrauma (U.S. Pat. Nos. 6,271,263 and 6,277,886) and from neurodegenerative diseases (U.S. Pat. No. 6,277,886). Neurotrauma is the damage to the central or peripheral nervous system caused by a traumatic event, such as head trauma, spinal trauma, neurotoxic injury, stroke, ischemia, hypoxia, anoxia (U.S. Pat. No. 6,277,886), or eye injury. Neurodegenerative diseases are diseases in which there is degeneration of the central or peripheral nervous system, such as multiple sclerosis (Lampert, P. W.), amyotrophic lateral sclerosis (Amyotrophic Lateral Sclerosis Information Page), Alzheimer's disease, dementia (U.S. Pat. Nos. 6,316,504 and 6,451,306), Parkinson's disease (U.S. Pat. Nos. 6,395,546 and 6,451,306), acute disseminated encephalomyelitis, adrenleukodystrophy, adrenomyeloneuropathy, Leber's hereditary optic atrophy, Human Lymphotrophic T-cell Virus (HTLV)-associated myelopathy, acute viral encephalitis, aseptic meningitis (Merck Manual), optic neuritis (McKhann), glaucoma (U.S. Pat. No. 6,444,676), and neuropathy.

In addition, providing neuroprotection encompasses providing protection of the central or peripheral nervous system from toxic levels of glutamate. Diseases which have been associated with toxic levels of glutamate include ALS (Ask the pharmacist: Common questions asked about Rilutek®), Huntington's disease (Ashizawa) and systemic lupus erythematosus (Beckman).

Providing neuroprotection also includes providing protection against toxic levels of monoamine oxidase-B (MAO-B) activity. Diseases and conditions which have been associated with toxic levels of monoamine oxidase-B are Parkinson's disease (Monoamine oxidase B inhibitors. Current status and future potential; Rationale for (−)deprenyl (Selegiline) therapy in Parkinson's disease and Alzheimer's disease; Rodriguez-Gomez et al.), Alzheimer's disease (Rationale for (−)deprenyl (Selegiline) therapy in Parkinson's disease and Alzheimer's disease; Monoamine oxidase B inhibitors. Current status and future potential; Potential applications for monoamine oxidase B inhibitors), memory disorders (The interaction of L-deprenyl and scopolamine on spatial learning/memory in rats), depression (Merck Manual of Diagnosis and Therapy; L-Deprenyl, a selective monoamine oxidase type-b inhibitor in endogenous depression; Potential applications for monoamine oxidase B inhibitors), panic, post-traumatic stress disorder (PTSD), sexual dysfunction, attention deficit and hyperactivity syndrome (ADHD)(Potential applications for monoamine oxidase B inhibitors), attention deficit disorder (Kleywegt), and Tourette's syndrome (Treatment of Tourette's: Overview).

Formulations of the invention suitable for oral administration may be in the form of capsules, pills, tablets, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the active compound or compounds.

In solid dosage forms of the invention for oral administration (capsules, tablets, pills, dragees, powders, granules and the like), the active ingredient(s) is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, calcium phosphate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers—in soft and hard-filled gelatin capsules using such excipients as lactose or milk sugars, as well as high molecular weight polyethylene glycols and the like.

Liquid dosage forms for oral administration of the active ingredients include pharmaceutically acceptable emulsions, microemulsions, solutions, suspensions, syrups and elixirs. In addition to the active ingredient(s), the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions, particularly those comprising glatiramer acetate, may also include human adjuvants or carriers known to those skilled in the art. Such adjuvants include complete Freund's adjuvant and incomplete Freund's adjuvant. The compositions may also comprise wetting agents, emulsifying and suspending agents, sweetening, flavoring, coloring, perfuming and preservative agents.

Glatiramer acetate may be formulated into pharmaceutical compositions with pharmaceutically acceptable carriers, such as water or saline and may be formulated into eye drops. Glatiramer acetate may also be formulated into delivery systems, such as matrix systems.

The subject invention provides a method of treating a subject afflicted with a form of multiple sclerosis comprising periodically administering to the subject an amount of glatiramer acetate and an amount of 2-amino-6-trifluoromethoxybenzathiazole, wherein the amounts when taken together are effective to alleviate a symptom of the form of multiple sclerosis in the subject so as to thereby treat the subject.

In one embodiment, the form of multiple sclerosis is relapsing-remitting multiple sclerosis.

In another embodiment, the subject is a human being.

In a further embodiment, each of the amount of glatiramer acetate when taken alone, and the amount of 2-amino-6-trifluoromethoxybenzathiazole when taken alone is effective to alleviate the symptom of the form of multiple sclerosis.

In an embodiment, either the amount of glatiramer acetate when taken alone, the amount of 2-amino-6-trifluoromethoxybenzathiazole when taken alone or each such amount when taken alone is not effective to alleviate the symptom of the form of multiple sclerosis.

In yet another embodiment, the symptom is the frequency of relapses, the frequency of clinical exacerbation, or the accumulation of physical disability.

In one embodiment, the amount of glatiramer acetate may be 10 to 80 mg; or 12 to 70 mg; or 14 to 60 mg; or 16 to 50 mg; or 18 to 40 mg; or 20 to 30 mg; or 20 mg. For each amount of glatiramer acetate, the amount of 2-amino-6-trifluoromethoxybenzathiazole may be 25 to 75 mg; or 35 to 65 mg; or 45 to 55 mg; or 50 mg.

Alternatively, the amount of glatiramer acetate may be in the range from 10 to 600 mg/week; or 100 to 550 mg/week; or 150 to 500 mg/week; or 200 to 450 mg/week; or 250 to 400 mg/week; or 300 to 350 mg/week; or 300 mg/week.

In another embodiment, the amount of glatiramer acetate may be in the range from 50 to 150 mg/day; or 60 to 140 mg/day; or 70 to 130 mg/day; or 80 to 120 mg/day; or 90 to 110 mg/day; or 100 mg/day.

Alternatively, the amount of glatiramer acetate may be in the range from 10 to 80 mg/day; or 12 to 70 mg/day; or 14 to 60 mg/day; or 16 to 50 mg/day; or 18 to 40 mg/day; or 19 to 30 mg/day; or 20 mg/day.

In one embodiment, the periodic administration of glatiramer acetate is effected daily.

In another embodiment, the periodic administration of glatiramer acetate is effected twice daily at one half the amount.

In an additional embodiment, the periodic administration of glatiramer acetate is effected once every 3 to 11 days; or once every 5 to 9 days; or once every 7 days; or once every 24 hours.

For each administration schedule of glatiramer acetate, the 2-amino-6-trifluoromethoxybenzathiazole may be administered once every 8 to 16 hours; or once every 10 to 14 hours; or once every 12 hours.

In an embodiment, the periodic administration of 2-amino-6-trifluoromethoxybenzathiazole is effected at least one hours before or at least two hours after a meal.

In a further embodiment, the administration of the glatiramer acetate substantially precedes the administration of the 2-amino-6-trifluoromethoxybenzathiazole.

In an added embodiment, the administration of the 2-amino-6-trifluoromethoxybenzathiazole substantially precedes the administration of the glatiramer acetate.

In one embodiment, the glatiramer acetate and the 2-amino-6-trifluoromethoxybenzathiazole may be administered for a period of time of at least 4 days. In a further embodiment, the period of time may be 5 days to 5 years; or 10 days to 3 years; or 2 weeks to 1 year; or 1 month to 6 months; or 3 months to 4 months. In yet another embodiment, the glatiramer acetate and the 2-amino-6-trifluoromethoxybenzathiazole may be administered for the lifetime of the subject.

The administration of 2-amino-6-trifluoromethoxybenzathiazole or glatiramer acetate may each independently be oral, nasal, pulmonary, parenteral, intravenous, intra-articular, transdermal, Intradermal, subcutaneous, topical, intramuscular, rectal, intrathecal, intraocular, buccal or by gavage. For 2-amino-6-trifluoromethoxybenzathiazole, the preferred route of administration is oral or by gavage. The preferred route of administration for glatiramer acetate is subcutaneous or oral. One of skill in the art would recognize that doses at the higher end of the range may be required for oral administration.

In one embodiment, the administration of the glatiramer acetate may be subcutaneous, intraperitoneal, intravenous, intramuscular, intraocular or oral and the administration of the 2-amino-6-trifluoromethoxybenzathiazole may be oral. In another embodiment, the administration of the glatiramer acetate may be subcutaneous and the administration of the 2-amino-6-trifluoromethoxybenzathiazole may be oral.

The subject invention also provides a package comprising
i) a first pharmaceutical composition comprising an amount of glatiramer acetate and a pharmaceutically acceptable carrier;
ii) a second pharmaceutical composition comprising an amount of 2-amino-6-trifluorormethoxybenzothiazole and a pharmaceutically acceptable carrier; and
iii) instructions for use of the first and second pharmaceutical compositions together to alleviate a symptom of a form of multiple sclerosis in a subject.

In an embodiment of the package, the amount of glatiramer acetate may be in the range from 10 to 600 mg; or 100 to 550 mg; or 150 to 500 mg; or 200 to 450 mg; or 250 to 400 mg; or 300 to 350 mg; or 300 mg.

In another embodiment of the package, the amount of glatiramer acetate may be in the range from 10 to 80 mg; or 12 to 70 mg; or 14 to 60 mg; or 16 to 50 mg; or 18 to 40 mg; or 19 to 30 mg; or 20 mg.

Alternatively, the amount of glatiramer acetate in the package may be in the range from 50 to 150 mg; or 60 to 140 mg; or 70 to 130 mg; or 80 to 120 mg; or 90 to 110 mg; or 100 mg.

For each amount of glatiramer acetate in the package, the amount of 2-amino-6-trifluoromethoxybenzathiazole in the package may be 25-75 mg; or 35-65 mg; or 45-55 mg; or 50 mg.

The subject invention further provides a pharmaceutical composition comprising an amount of glatiramer acetate and an amount of 2-amino-6-trifluorormethoxybenzothiazole, wherein the amounts when taken together are effective to alleviate a symptom of a form of multiple sclerosis in a subject.

In one embodiment of the pharmaceutical composition, each of the amount of glatiramer acetate when taken alone and the amount of 2-amino-6-trifluoromethoxybenzathiazole when taken alone is effective to alleviate the symptom of multiple sclerosis.

In another embodiment of the pharmaceutical composition, either of the amount of glatiramer acetate when taken alone, or the amount of 2-amino-6-trifluoromethoxybenzathiazole when taken alone or each such amount when taken alone is not effective to alleviate the symptom of multiple sclerosis.

The subject invention further provides a pharmaceutical combination comprising separate dosage forms of an amount of glatiramer acetate and an amount of 2-amino-6-trifluorormethoxybenzothiazole, which combination is useful to alleviate a symptom of a form of multiple sclerosis in a subject.

In an embodiment of the pharmaceutical combination, each of the amount of glatiramer acetate when taken alone and the amount of 2-amino-6-trifluoromethoxybenzathiazole when taken alone is effective to alleviate the symptom of multiple sclerosis.

In an additional embodiment of the pharmaceutical combination, either of the amount of glatiramer acetate when taken alone, the amount of 2-amino-6-trifluoromethoxybenzathiazole when taken alone or each such amount when taken alone is not effective to alleviate the symptom of multiple sclerosis.

In a further embodiment, the pharmaceutical combination may be for simultaneous, separate or sequential use to treat the form of multiple sclerosis in the subject.

Formulations of the invention suitable for oral administration may be in the form of capsules, pills, tablets, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the active compound or compounds.

In solid dosage forms of the invention for oral administration (capsules, tablets, pills, dragees, powders, granules and the like), the active ingredient(s) is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, calcium phosphate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the active ingredients include pharmaceutically acceptable emulsions, microemulsions, solutions, suspensions, syrups and elixirs. In addition to the active ingredient(s), the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions, particularly those comprising glatiramer acetate, may also include human adjuvants or carriers known to those skilled in the art. Such adjuvants include complete Freund's adjuvant and incomplete Freund's adjuvant. The compositions may also comprise wetting agents, emulsifying and suspending agents, sweetening, flavoring, coloring, perfuming and preservative agents.

Glatiramer acetate may be formulated into pharmaceutical compositions with pharmaceutically acceptable carriers, such as water or saline and may be formulated into eye drops. Glatiramer acetate may also be formulated into delivery systems, such as matrix systems.

The subject invention further provides a method of treating a subject afflicted with amyotrophic lateral sclerosis comprising periodically administering to the subject an amount of glatiramer acetate and an amount of 2-amino-6-trifluoromethoxybenzathiazole, wherein the amounts when taken together are effective to alleviate a symptom of amyotrophic lateral sclerosis in the subject so as to thereby treat the subject.

In one embodiment, the subject is a human being.

In a further embodiment, each of the amount of glatiramer acetate when taken alone, and the amount of 2-amino-6-trifluoromethoxybenzathiazole when taken alone is effective to alleviate the symptom of amyotrophic lateral sclerosis.

In an embodiment, either the amount of glatiramer acetate when taken alone, the amount of 2-amino-6-trifluoromethoxybenzathiazole when taken alone or each such amount when taken alone is not effective to alleviate the symptom of amyotrophic lateral sclerosis.

In yet another embodiment, the symptom is twitching, cramping, loss of motor control, or difficulties in speaking, swallowing or breathing.

In one embodiment, the amount of glatiramer acetate may be 10 to 80 mg; or 12 to 70 mg; or 14 to 60 mg; or 16 to 50 mg; or 18 to 40 mg; or 20 to 30 mg; or 20 mg. For each amount of glatiramer acetate, the amount of 2-amino-6-trifluoromethoxybenzathiazole may be 25 to 75 mg; or 35 to 65 mg; or 45 to 55 mg; or 50 mg.

Alternatively, the amount of glatiramer acetate may be in the range from 10 to 600 mg/week; or 100 to 550 mg/week; or 150 to 500 mg/week; or 200 to 450 mg/week; or 250 to 400 mg/week; or 300 to 350 mg/week; or 300 mg/week.

In another embodiment, the amount of glatiramer acetate may be in the range from 50 to 150 mg/day; or 60 to 140 mg/day; or 70 to 130 mg/day; or 80 to 120 mg/day; or 90 to 110 mg/day; or 100 mg/day.

Alternatively, the amount of glatiramer acetate may be in the range from 10 to 80 mg/day; or 12 to 70 mg/day; or 14 to 60 mg/day; or 16 to 50 mg/day; or 18 to 40 mg/day; or 19 to 30 mg/day; or 20 mg/day.

In one embodiment, the periodic administration of glatiramer acetate is effected daily.

In another embodiment, the periodic administration of glatiramer acetate is effected twice daily at one half the amount.

In an additional embodiment, the periodic administration of glatiramer acetate is effected once every 3 to 11 days; or once every 5 to 9 days; or once every 7 days; or once every 24 hours.

For each administration schedule of glatiramer acetate, the 2-amino-6-trifluoromethoxybenzathiazole may be administered once every 8 to 16 hours; or once every 10 to 14 hours; or once every 12 hours.

In an embodiment, the periodic administration of 2-amino-6-trifluoromethoxybenzathiazole is effected at least one hour before or at least two hours after a meal.

In a further embodiment, the administration of the glatiramer acetate substantially precedes the administration of the 2-amino-6-trifluoromethoxybenzathiazole.

In an added embodiment, the administration of the 2-amino-6-trifluoromethoxybenzathiazole substantially precedes the administration of the glatiramer acetate.

In one embodiment, the glatiramer acetate and the 2-amino-6-trifluoromethoxybenzathiazole may be administered for a period of time of at least 4 days. In a further embodiment, the period of time may be 5 days to 5 years; or 10 days to 3 years; or 2 weeks to 1 year; or 1 month to 6 months; or 3 months to 4 months. In yet another embodiment, the glatiramer acetate and the 2-amino-6-trifluoromethoxybenzathiazole may be administered for the lifetime of the subject.

The administration of 2-amino-6-trifluoromethoxybenzathiazole or glatiramer acetate may each independently be oral, nasal, pulmonary, parenteral, intravenous, intra-articular, transdermal, intradermal, subcutaneous, topical, intramuscular, rectal, intrathecal, intraocular, buccal or by gavage. For 2-amino-6-trifluoromethoxybenzathiazole, the preferred route of administration is oral or by gavage. The preferred route of administration for glatiramer acetate is subcutaneous or oral. One of skill in the art would recognize that doses at the higher end of the range may be required for oral administration.

In one embodiment, the administration of the glatiramer acetate may be subcutaneous, intraperitoneal, intravenous, intramuscular, intraocular or oral and the administration of the 2-amino-6-trifluoromethoxybenzathiazole may be oral. In another embodiment, the administration of the glatiramer acetate may be subcutaneous and the administration of the 2-amino-6-trifluoromethoxybenzathiazole may be oral.

The subject invention also provides a package comprising
i) a first pharmaceutical composition comprising an amount of glatiramer acetate and a pharmaceutically acceptable carrier;
ii) a second pharmaceutical composition comprising an amount of 2-amino-6-trifluorormethoxybenzothiazole and a pharmaceutically acceptable carrier; and
iii) instructions for use of the first and second pharmaceutical compositions together to alleviate a symptom of amyotrophic lateral sclerosis in a subject.

In an embodiment of the package, the amount of glatiramer acetate may be in the range from 10 to 600 mg; or 100 to 550 mg; or 150 to 500 mg; or 200 to 450 mg; or 250 to 400 mg; or 300 to 350 mg; or 300 mg.

In another embodiment of the package, the amount of glatiramer acetate may be in the range from 10 to 80 mg; or 12 to 70 mg; or 14 to 60 mg; or 16 to 50 mg; or 18 to 40 mg; or 19 to 30 mg; or 20 mg.

Alternatively, the amount of glatiramer acetate in the package may be in the range from 50 to 150 mg; or 60 to 140 mg; or 70 to 130 mg; or 80 to 120 mg; or 90 to 110 mg; or 100 mg.

For each amount of glatiramer acetate in the package, the amount of 2-amino-6-trifluoromethoxybenzathiazole in the package may be 25-75 mg; or 35-65 mg; or 45-55 mg; or 50 mg.

The subject invention further provides a pharmaceutical composition comprising an amount of glatiramer acetate and an amount of 2-amino-6-trifluorormethoxybenzothiazole, wherein the amounts when taken together are effective to alleviate a symptom of amyotrophic lateral sclerosis in a subject.

In one embodiment of the pharmaceutical composition, each of the amount of glatiramer acetate when taken alone and the amount of 2-amino-6-trifluoromethoxybenzathiazole when taken alone is effective to alleviate the symptom of amyotrophic lateral sclerosis.

In another embodiment of the pharmaceutical composition, either of the amount of glatiramer acetate when taken alone, or the amount of 2-amino-6-trifluoromethoxybenzathiazole when taken alone or each such amount when taken alone is not effective to alleviate the symptom of amyotrophic lateral sclerosis.

The subject invention further provides a pharmaceutical combination comprising separate dosage forms of an amount of glatiramer acetate and an amount of 2-amino-6-trifluorormethoxybenzothiazole, which combination is useful to alleviate a symptom of amyotrophic lateral sclerosis in a subject.

In an embodiment of the pharmaceutical combination, each of the amount of glatiramer acetate when taken alone and the amount of 2-amino-6-trifluoromethoxybenzathiazole when taken alone is effective to alleviate the symptom of amyotrophic lateral sclerosis.

In an additional embodiment of the pharmaceutical combination, wherein either of the amount of glatiramer acetate when taken alone, the amount of 2-amino-6-trifluoromethoxybenzathiazole when taken alone or each such amount when taken alone is not effective to alleviate the symptom of amyotrophic lateral sclerosis.

In a further embodiment, the pharmaceutical combination may be for simultaneous, separate or sequential use to treat amyotrophic lateral sclerosis in the subject.

Formulations of the invention suitable for oral administration may be in the form of capsules, pills, tablets, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the active compound or compounds.

In solid dosage forms of the invention for oral administration (capsules, tablets, pills, dragees, powders, granules and the like), the active ingredient(s) is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, calcium phosphate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the active ingredients include pharmaceutically acceptable emulsions, microemulsions, solutions, suspensions, syrups and elixirs. In addition to the active ingredient(s), the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions, particularly those comprising glatiramer acetate, may also include human adjuvants or carriers known to those skilled in the art. Such adjuvants include complete Freund's adjuvant and incomplete Freund's adjuvant. The compositions may also comprise wetting agents, emulsifying and suspending agents, sweetening, flavoring, coloring, perfuming and preservative agents.

Glatiramer acetate may be formulated into pharmaceutical compositions with pharmaceutically acceptable carriers, such as water or saline and may be formulated into eye drops. Glatiramer acetate may also be formulated into delivery systems, such as matrix systems.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

### Example 1

### Glutamate Toxicity

Under normal circumstances, glutamate functions as an essential neurotransmitter. However, when glutamate levels rise above the normal level, glutamate becomes toxic. Elevated glutamate levels and the resultant toxicity are implicated in many diseases, as discussed in the Background of the Invention.

Procedure

60 mice are injected with glutamate (0.2 M) to induce retinal ganglion cell (RGC) death. As shown in Table 1, mice are immunized with glatiramer acetate, riluzole or both prior to glutamate injection. Glatiramer acetate is given s.c. (subcutaneously), 100 μl/mouse, with or without adjuvant. Glatiramer acetate can also be administered orally, with or without adjuvant. Glatiramer acetate may be administered over several doses before the glutamate challenge or may be administered simultaneously with glutamate. Riluzole is administered by gavage in 4 doses of 10 mg/kg each. The control animals receive PBS without any active agents, although any vehicle can be used.

TABLE 1
Administration Protocol

| Experimental | Glatiramer | | |
|---|---|---|---|
| Groups | Acetate | Riluzole | Glutamate |
| A | X | X | X |
| B | X | — | X |
| C | — | X | X |
| D | — | — | X |
| E | | | |

7 days after glutamate injection, mice are sacrificed, retinas are excised and surviving RGCs are counted. Following immunization, in vitro cellular response to specific activators, such as glatiramer acetate, and to non-specific activators, such as Con-A, PHA, etc., are assessed.

Results The administration of only glatiramer acetate (Group B) or only riluzole (Group C) prior to glutamate challenge raises the number of surviving RGCs above that of the control group that receives only glutamate (Group E). When both glatiramer acetate and riluzole are administered before the glutamate administration (Group A), the number of surviving RGCs is comparable to or greater than the number of surviving RGCs in Group B (glatiramer acetate alone) or Group C (riluzole alone).

### Example 2

### MPTP-Induced Dopaminergic Neurotoxicity

MPTP is a neurotoxin that damages nigrostriatal dopaminergic neurons in several mammalian species, including mice, and produces a Parkinsonian syndrome in humans and primates. A crucial initial step in the mechanism of its neurotoxicity involves conversion of MPTP to its toxic metabolite 1-methyl-4-phenyl pyridinium ion (MPP+). This reaction is catalyzed by the enzyme MAO-B and probably takes place outside of dopaminergic neurons, mainly in glia (U.S. Pat. No. 6,316,504).

Procedure

i) Animals

Mice (C57B16 males weighing 20-25 g, 6-8 weeks of age) are obtained from Harlan (Jerusalem) and housed 5 per cage for 1 week before treatment. Standard mouse chow and water is supplied ad libitum. Room lighting is 12 hours light, 12 hours dark; lights on at 7:00 AM. The cages are maintained in a locked room in the animal house, accessible only to personnel familiar with the safety rules for MPTP administration, and wearing appropriate protective clothing.

ii) Materials

Glatiramer Acetate

Riluzole

MPTP hydrochloride, Sigma cat. #M0896, lot #89H4702

PBS: phosphate-buffered saline (g per liter): NaCl 8, KCl 0.2, Na₂HPO₄ 1.44, KH₂PO₄ 0.24; pH 7.4 (materials from Sigma, Israel) sterile saline: NaCl, 0.9%

iii) Glatiramer Acetate/Riluzole Administration

Table 2 presents the dosing schedule for the groups of mice (10 mice per group). 7 days before MPTP administration, the mice in Groups B, D and F are injected s.c. with 0.2 ml of a solution of glatiramer acetate (0.5 mg/ml) in PBS. This injection correlates to 100 μg/mouse. This dose is used because it has been used in other studies (glutamate toxicity, retinal intraocular pressure (IOP) model). Riluzole (10 mg/kg) is administered by gavage to the mice in Groups B, E and F 30 minutes before each of 4 MPTP injections. The members of control group A are injected s.c. with 0.2 ml of PBS and do not receive MPTP. The mice in control group B are injected s.c. with glatiramer acetate (100 μg/mouse) and given riluzole (10 mg/kg) by gavage, and saline instead of MPTP. The mice in control group C are injected s.c. with 0.2 ml of PBS followed by MPTP administration as outlined below.

TABLE 2
Administration Protocol

| Experimental | | | |
|---|---|---|---|
| Groups | GA | Riluzole | MPTP |
| A | | | |
| B | X | X | |
| C | | | X |
| D | X | | X |
| E | | X | X |
| F | X | X | X |

iv) MPTP Administration

For the mice that have received glatiramer acetate, 7 days after glatiramer acetate immunization, cage bedding is changed and MPTP treatment is commenced. MPTP is obtained from Sigma in the form of 10 mg pre-weighed vials. The vial contents are dissolved in 2 ml sterile saline solution (0.9%) to yield a solution containing 5 mg/ml MPTP. Each mouse is injected intraperitoneally (i.p.) with 0.1 ml of this solution per 20 g body weight, to give a dose of 25 mg per kg body weight. A total of 4 injections are given to each mouse, on each of 4 successive days, injections being made between 10 AM and 12 noon. Control animals receive the same volume of sterile saline (0.1 ml per 20 g body weight).

v) Precautions During Use of MPTP

Personnel wear disposable laboratory coats, disposable latex rubber gloves and carbon filter face masks for the administration of MPTP. Cage bedding and all utensils in contact with MPTP is exposed to a 20% Clorox® solution (commercial bleach) for 30 minutes before containment in biohazard grade disposable nylon bags.

vi) Clinical Assessment

Mice are observed for 30 minutes following each injection of MPTP.

vii) Determination of Striatal Dopamine

Mice are sacrificed by cervical dislocation 7 days after the first MPTP injection. Brains are removed and cooled on ice. Each whole brain is placed on its dorsal surface on an ice-cooled glass plate. A coronal cut is made with a razor blade at the level of the optic chiasm. Striata is dissected from the frontal part of the brain and snap-frozen in liquid nitrogen, then stored at −70° C. until homogenization.

Immediately prior to homogenization, the tissue is weighed. The vials are removed from the −70° C. refrigerator and quickly placed in a liquid nitrogen container. Taking care to keep the cap closed, each vial is then separately removed from the liquid nitrogen container. The frozen tissue is then quickly and carefully removed from the vial and placed on a pre-weighed dish in the analytical balance. The tissue is then weighed.

For homogenization, the tissue is placed in an Ependorff tube to which 300 μl of a 0.1 M perchlorate solution containing 2 mM sodium metabisulfite and 0.25 mM ethylenediaminetetracetic acid (EDTA) is added. The tissue is homogenized for 30 seconds in ice using an Ependorff miniature homogenizer. Then, 300 μl perchlorate is added (0.6 ml for more than 20 mg tissue).

The tissue is then centrifuged at 13000 g for 5-7 minutes and the supernatant is decanted for catecholamine determination. If the analysis is not done on the same day, the samples can be frozen at −70° C. and centrifuged again after thawing before analysis.

To detect the presence and amounts of dopamine, DOPAC and HVA, a hypersil H3ODS column (packing 3 mm, 4.6 mm diameter, 12.5 cm long) is used. The mobile phase is composed of NaH₂PO₄ 100 mM, octan-1-sulphonic acid 1.5 mM, disodium ethylenediaminetetracetic acid 250 μM, methanol 2.3%, and acetonitrile 4% in HPLC grade deionized water, at a flow rate of 1.0 ml min⁻¹. Compounds are detected with an ESA Coulochem model 5014 electrochemical detector (Bedford, Mass., USA). Column eluates are initially oxidized by an ESA guard cell (model 5020) at +300 mV, then reduced at +60 mV at detector 1 and measured at detector 2 at −350 mV. The catecholamines are compared to standards (10⁻⁷M prepared in perchlorate). Dilution of the sample (1:10) for the dopamine determination might be needed. Levels of dopamine and metabolites are expressed in terms of pmol per mg tissue (frozen weight).

Results

In the absence of MPTP (Groups A and B), striatal dopamine levels are around 60-90 pmol/mg tissue. The administration of MPTP reduces the striatal dopamine levels to around 25-45 pmol/mg tissue (Group C). Immunization with glatiramer acetate alone (Group D) prior to the administration of MPTP increases the striatal dopamine levels. The administration of riluzole alone (Group E) before MPTP challenge increases the striatal dopamine levels. In Group F, the combined administration of glatiramer acetate and riluzole before injection of MPTP results striatal dopamine levels that are comparable to or greater than the striatal dopamine levels of Group D (glatiramer acetate alone) or Group E (riluzole alone).

### Example 3

### Experimental Model for Amyotrophic Lateral Sclerosis

Procedure

Transgenic mice carrying multiple copies of the human G93A Cu/Zn SOD mutation are considered to be the best model system for anterior horn cell degenerations such as amyotrophic lateral sclerosis (Ludolph et al.; Gurney et al. 1994 and 1996).

i) Animals

Transgenic mice overexpressing human Cu/Zn-SOD G93A mutations ((B6SJL-TgN (SOD1-G93A) 1 Gur) and non-transgenic B6/SJL mice are purchased from Jackson Laboratories (Bar Harbor, Me., USA). The second generation of G1H mice are used.

ii) Treatment Protocol

The SOD1 transgenic mice are treated in five groups (N=15) with two dosages of glatiramer acetate alone or in combination with riluzole. A group of 15 mice serve as controls. Treatment protocols for the six groups were the following:
Group I: Low dose glatiramer acetate
Group II: High dose glatiramer acetate
Group III: Riluzole 30 mg/kg per day
Group IV: Low dose glatiramer acetate and riluzole 30 mg/kg per day
Group V: High dose glatiramer acetate 2.0 mg/kg and riluzole 30 mg/kg per day
Group VI: Controls (placebo)

The drugs are administered in drinking water starting at 60 days of age.

iii) Survival

The clinical condition of the mice is monitored daily starting at 40 days. The onset of clinical signs is scored by examining the mice for tremors and/or shaking of the limbs, the position of one or both hind limbs (hanging rather than splaying out) when the mice are suspended in the air by their tail. The age of clinical onset was determined by the age (days) at which loss of splay or tremors of hind limbs is observed. The loss of righting reflex determines the end stage of the disease. The mice are sacrificed if they could not right themselves within 30 seconds when placed on either side on a flat surface.

iv) Behavior and Weight Assessment

Mice are observed daily (including weekends) and weighed weekly. Motor performance is assessed from 40 days of age using the rotarod apparatus to measure the night activity of the mice from 8 p.m.-8 a.m. (LMTB, Berlin).

v) Neuropathological Studies

Mice were perfused transcardially with 4% paraformaldehyde/0.9% NaCl solution, brains and whole spinal cord were dissected out, frozen in liquid nitrogen and cut in transverse sections of 20 μm on a sliding microtome. Sections of brainstem and spinal cord were stained with HE, toluidine blue (semi-thin sections, 0.5 μm), and immunohistochemistry to label astrocytes (GFAP), cholinergic motoneurons (ChAT) and dopaminergic cells (TH).

Results

i) Survival

The primary end point of this study is survival. Animals treated with the low dose of glatiramer acetate alone show an increase in life span which is not statistically significant. The high dose of glatiramer acetate produces a statistically significant increase in life span. Both combinations of riluzole and glatiramer acetate result in an increase in life span which is comparable to or greater than the increase in life span in the groups receiving only glatiramer acetate.

ii) Behavior

The results of measurements of running wheel activity in the 6 groups are largely complementary to the survival data. Statistical analysis show that glatiramer acetate-treated (at both dosages) and riluzole-treated animals are more active during the course of treatment and maintain their motor activity longer than controls. Treatment with riluzole combined with either low-dose or high-dose glatiramer acetate results in activity comparable to or greater than treatment with riluzole alone or glatiramer acetate alone.

iii) Neuropathological Studies

Neuropathological examination of the spinal cord shows the typical features of G93A-associated damage; i.e. loss of motor neurons, astrocytosis and extensive vacuolation of the spinal grey matter. At the light-microscopic level, there are no differences in the degree of neuronal loss, the morphology of astrocytosis or the extent of vacuolisation between animals. This lack of effect on histopathology may be due to the fact that all the animals are sacrificed at terminal stages of the disease.

### Example 4

### Clinical Trial for Alzheimer's Disease

Procedure

A double-blind clinical trial is conducted on patients with Alzheimer's disease. Glatiramer acetate and riluzole (50 mg b.i.d. (twice daily), oral) are administered to 20 patients selected at random. Another group of 20 patients receive placebo following the same dosage schedule as the treatment group. Mental function (ability to process information and short-term and long-term memory) is assessed monthly for a year by standard clinical and laboratory tests. The inclusion and exclusion criteria for patients are determined according to medically accepted standards.

Results

In the clinical and laboratory tests, the group receiving glatiramer acetate and riluzole shows improved ability to process information and ability to recall information in the short term and the long-term as compared to the group receiving placebo.

### Example 5

### Clinical Trial for Parkinson's Disease

Procedure

A double-blind clinical trial is conducted on patients with Parkinson's disease. Sinemet® (Carbidopa-Levodopa) is administered to each patient according to the dosing schedule determined by medically acceptable standards to be most appropriate for that patient. In addition to Sinemet®, glatiramer acetate and riluzole (50 mg b.i.d. (twice daily), oral) are administered to 20 patients selected at random. Another group of 20 patients receive a placebo according to the same dosing schedule, in addition to Sinemet®. The following parameters are measured at entry into the trial and assessed monthly for a year thereafter: measurement on Alzheimer's Disease Assessment Scale—Cognitive Subscale (ADAS-cog), cognitive function (ability to process information and short-term and long-term memory), daily activities, and mood. The inclusion and exclusion criteria for patients are determined according to medically accepted standards.

Results

The administration of both glatiramer acetate and riluzole along with Sinemet® produces, results that are comparable to or greater than those of the control groups receiving only Sinemet® for all parameters tested—measurement on Alzheimer's Disease Assessment Scale—Cognitive Subscale (ADAS-cog), cognitive function (ability to process information and short-term and long-term memory), daily activities, and mood.

### Example 6

### Clinical Trial for Glaucoma

Procedure

Separate double-blind clinical trial are conducted on patients with the following types of glaucoma: i) acute close-angle glaucoma; ii) chronic close-angle glaucoma; iii) acute open-angle glaucoma; iv) chronic open-angle glaucoma

In each trial, glatiramer acetate and riluzole (50 mg b.i.d. (twice daily), oral) are administered to 20 patients selected at random. Another group of 20 patients receive placebo following the same dosage schedule as the treatment group. Intraocular pressure (IOP) is measured and a visual field test is performed at entry into the trial and assessed every two weeks for a year. The inclusion and exclusion criteria for patients are determined according to medically accepted standards.

Results

In each trial, the administration of glatiramer acetate and riluzole results in a comparable or greater reduction of intraocular pressure than the administration of placebo. Additionally, the patients receiving glatiramer acetate and riluzole showed a comparable or greater improvement in the visual field test in comparison to the patients receiving placebo.

### Example 7

### Clinical Trial for Head Injury

Procedure

A double-blind clinical trial is conducted on patients with head injury Glatiramer acetate and riluzole (50 mg b.i.d. (twice daily), oral) are administered to 20 patients selected at random. Another group of 20 patients receive placebo following the same dosage schedule as the treatment group. The following parameters are determined at entry into the trial and assessed every two to four weeks for the first three months, monthly for the next three months and then every two months for the next six months: mental function (vocabulary, problem solving, memory), mood, motor coordination, development of epilepsy, and length of survival. The inclusion and exclusion criteria for patients are determined according to medically accepted standards.

Results

In comparison to the controls, patients being treated with glatiramer acetate and riluzole exhibit comparable or greater mental function (vocabulary, problem solving, memory) and motor coordination. In addition, the treatment group shows comparable or better moods and comparable or greater survival time than the controls. The patients who receive glatiramer acetate and riluzole also demonstrate a comparable or greater reduction in development of epilepsy than the controls.

### Example 8

### Clinical Trial of Spinal Cord Injury

Procedure

A double-blind clinical trial is conducted on patients with spinal cord injury. Glatiramer acetate and riluzole (50 mg b.i.d. (twice daily), oral) are administered to 20 patients selected at random. Another group of 20 patients receive placebo following the same dosage schedule as the treatment group. The following parameters are determined at entry into the trial and assessed every two to four weeks for the first three months, monthly for the next three months and then every two months for the next six months: muscular spasms and ability to control affected muscles, including motor function, sexual function and bladder and bowel control. The inclusion and exclusion criteria for patients are determined according to medically accepted standards.

Results

The administration of glatiramer acetate and riluzole result in comparable or greater ability to control affected muscles (motor function, sexual function and bladder and bowel control) than the administration of placebo. Treatment with glatiramer acetate and riluzole also results in a comparable or greater reduction in muscular spasms in comparison to placebo treatment.

### Example 9

### Clinical Trial of Stroke

Experiment 9i): Ischemic Stroke—Clinical Procedure

A double-blind clinical trial is conducted on patients with ischemia. Glatiramer acetate and riluzole (50 mg b.i.d. (twice daily), oral), and an anti-coagulant at its medically accepted dose are administered to 20 patients selected at random. Another group of 20 patients receive placebo and the anti-coagulant following the same dosage schedule as the treatment group. The following parameters are determined at entry into the trial and assessed every one to four weeks for the first three months and monthly for the next nine months: score on NIH stroke scale, cognitive function (ability to process information and short-term and long-term memory), and infract volume by MRI and CT scan. The inclusion and exclusion criteria for patients are determined according to medically accepted standards.

Experiment 9ii): Hemorrhagic Stroke—Clinical Procedure

The procedure of Experiment 101) is followed except that the patients are suffering from hemorrhagic stroke and an anti-coagulant is not administered.

Results of Experiments 9i) and 9ii)

In both clinical trials, patients receiving glatiramer acetate and riluzole show a comparable or greater score on the NIH stroke scale and cognitive function (ability to process information and short-term and long-term memory) as compared to the controls. Additionally, the administration of glatiramer acetate and riluzole results in a comparable or greater reduction volume of infracts than the controls.

### Example 10

### Clinical Trial of Anoxia/Hypoxia

Procedure

A double-blind clinical trial is conducted on patients suffering from axonia or hypoxia. Glatiramer acetate and riluzole (50 mg b.i.d. (twice daily), oral) are administered to 20 patients selected at random. Another group of 20 patients receive placebo following the same dosage schedule as the treatment group. Assessment of neurological function (Bayley II scale and other tests), behavior (HOME and NCAST) and MRI (qualitative and quantitative sensorimotor studies) are conducted on a monthly basis for a year. The inclusion and exclusion criteria for patients are determined according to medically accepted standards.

Results

In comparison to the controls, the patients receiving glatiramer acetate and riluzole exhibit comparable or greater neurological function and behavior. Additionally, the MRI images of the treatment group show a comparable or greater reduction in abnormalities than the control group.

### Example 11

### Clinical Trial of Demyelinating Disease

Separate double-blind clinical trial are conducted on patients with the following demyelinating diseases: i) acute disseminated encephalomyelitis; ii) adrenleukodystrophy; iii) adrenomyeloneuropathy; iv) Leber's hereditary optic atrophy; v) Human Lymphotrophic T-cell Virus 1 (HTLV-1)-associated myelopathy; vi) acute viral encephalitis; and vii) aseptic meningitis.

Procedure

In each trial, glatiramer acetate (20 mg/day, subcutaneous) and riluzole (50 mg b.i.d. (twice daily), oral) are administered to 20 patients selected at random. Another group of 20 patients receive glatiramer acetate (20 mg/day, subcutaneous) and placebo (50 mg b.i.d. (twice daily), oral). The following outcomes are measured: 1) Number of documented relapses; 2) Differences in mean change in EDSS (extended disability status scale); 3) Number of new T1 gadolinium (Gd) enhancing lesions; 5) Volume of T1-Gd enhancing lesions; 4) Total number of new T2 lesions; 5) Volume of T2 lesions; 6) Volume and number of hypointense lesions in T1 scans. Clinical assessments take place every 3 months (EDSS, T25W, AI). Lab tests and MRI are performed at the beginning of the study (baseline), then months 3, 6 and 12. The inclusion and exclusion criteria for patients are determined according to medically accepted standards.

Results

For all clinical trials, patients receiving glatiramer acetate and riluzole show a comparable or greater reduction in T1 and T2 Gd-enhancing lesions and other lesions in comparison to the controls. The administration of glatiramer acetate and riluzole results in a comparable or greater reduction in the number of relapses per year as compared to the controls.

### Example 12

### Clinical Trial of Multiple Sclerosis

The purpose of this trial is to compare the treatment of participants with relapsing-remitting multiple sclerosis (RR-MS) with COPAXONE® in combination with riluzole, with treatment with COPAXONE® in combination with placebo. The clinical objective is to evaluate the effect of treatments on MRI variables, clinical evaluations and immunological profile.

The design of this trial is a randomized, double-masked, 2-arm study of COPAXONE® in combination with riluzole versus COPAXONE® in combination with placebo for the treatment of relapsing-remitting multiple sclerosis. Twenty patients with RR-MS who meet the inclusion/exclusion criteria are enrolled per arm. Patients are randomized and receive either 20 mg SQ (subcutaneous) of COPAXONE® daily plus an oral dose of placebo daily or 20 mg SQ of COPAXONE® in combination with 50 mg riluzole every 12 hours. The riluzole is administered at least one hour before a meal or at least two hours after a meal.

Participant inclusion criteria are as follows: 1) men or women age 18 to 50 years; 2) RR-MS according to the guidelines from the International Panel on the Diagnosis of MS (McDonald et al.); 3) two separate documented relapses in the last two years; 4) active MRI with at least one gadolinium(Gd)-enhancing lesion in the MRI scan at screening; 5) EDSS (extended disability status scale) score between 1.0 and 5.0; 6) no relapse during screening period; 6) pre-treatment with COPAXONE® for at least three weeks, but no more than four weeks, prior to baseline visit; and 7) ability to understand and provide informed consent.

Participant exclusion criteria include the following: 1) normal brain MRI; 2) prior treatment with COPAXONE® other than the scheduled three to four week pretreatment prior to baseline visit; 3) previous treatment with immunomodulating agents such as interferon beta or IVIg for the last 6 months prior to entry; 4) previous use of immunosuppressive agents (including azathioprine) in the last 12 months prior study entry; 5) steroid treatment one month prior to entry; 6) women not willing to practice reliable methods of contraception; 7) pregnant or nursing women; 8) life threatening or clinically significant diseases; 9) history of alcohol and drug abuse within 6 months prior enrollment; 10) known history of sensitivity to Gd; 11) uncontrolled and uncontrollable head movements (tremor, tics, etc.), muscle spasms, significant urinary urgency and claustrophobia, which will prevent the subject from lying still during the MRI scan; and 12) participation in other investigational therapy in the last 90 days.

MRI scans are performed during the screening visit (for eligibility) and at months 5, 10, 11 and 12. Full physical and neurological examinations are performed at screening, baseline and at months 2, 5, 9 and 12. Safety laboratory is performed at screening baseline and at months 1, 2, 5, 9 and 12. In addition, blood Ca⁺ levels are monitored on the first and second months after baseline visit. The immunological profile is monitored at baseline and at months 1, 2, 4, and 5.

Primary efficacy endpoints include the following: 1) MRI variables as measured on months 10, 11, and 12; 2) total number and volume of T1 GD-enhanced lesions; 3) total number of new T2 lesions; and 4) total volume of T2 lesions. Secondary efficacy endpoints encompass the following: 1) changes in immunological parameters; and 2) PBMC proliferation in response to GA in vitro. The tertiary efficacy endpoints are as follows: 1) change from baseline in relapse rate and MS Functional Composite Score (MSFC); and 2) brain atrophy. Tolerability is evaluated with reference to the following: 1) percentage of subjects who discontinue the study; and 2) percentage of subjects who discontinue the study due to adverse events. Safety is evaluated with reference to 1) adverse event frequency and severity; 2) changes in vital signs and 3) clinical laboratory values.

Patients treated with the COPAXONE® and riluzole combination exhibit a comparable or greater reduction in T1 and T2 Gd-enhancing lesions and other lesions, as compared to the group receiving COPAXONE® and placebo. Additionally, the group receiving the COPAXONE® and riluzole combination demonstrate a comparable or greater reduction in the number of relapses per year as compared with the group receiving COPAXONE® and placebo.

### REFERENCES

U.S. Pat. No. 3,849,550, issued Nov. 19, 1974 (Teitelbaum, et al.).
U.S. Pat. No. 5,486,541, issued Jan. 23, 1996 (Sterling et al.).
U.S. Pat. No. 5,674,885, issued Oct. 7, 1997 (Boireau et al.).
U.S. Pat. No. 5,800,808, issued Sep. 1, 1998 (Konfino, et al.).
U.S. Pat. No. 5,858,964, issued Jan. 12, 1999 (Aharoni, et al.).
U.S. Pat. No. 5,981,589, issued Nov. 9, 1999 (Konfino, et al.).
U.S. Pat. No. 6,048,898, issued Apr. 11, 2000 (Konfino, et al.).
U.S. Pat. No. 6,054,430, issued Apr. 25, 2000 (Konfino, et al.)
U.S. Pat. No. 6,214,791, issued Apr. 10, 2001 (Amon, et al.).
U.S. Pat. No. 6,271,263, issued Aug. 7, 2001 (Herzig et al.).
U.S. Pat. No. 6,277,886, issued Aug. 21, 2001, (Levy et al.).
U.S. Pat. No. 6,316,504, issued Nov. 13, 2002 (Youdim et al.).
U.S. Pat. No. 6,395,546, issued May 28, 2002 (Zobel et al.).
U.S. Pat. No. 6,342,476, issued Jan. 29, 2002 (Konfino, et al.).
U.S. Pat. No. 6,362,161, issued Mar. 26, 2002 (Konfino et al.)
U.S. Pat. No. 6,444,676, issued Sep. 3, 2002 (Pang et al.).
U.S. Pat. No. 6,451,306, issued Sep. 17, 2002 (Tuszynski et al.).
Ashizawa, New Treatment Strategies, Huntington's disease workshop, Houston, Tex. <http://www.bcm.tmc.edu/neurol/struct/hunting/huntp8.html> (abstract).
Beckman, M., Antibody Destroys Neurons in Lupus, Science Now, Nov. 5, 2001<http://www.bric.postech.ac.kr/science/97now/01_{—}11now/011105a.html>.
Filippi, M. et al., Glatiramer acetate reduces the proportion of MS lesions evolving into black holes, Neurol., 2001, 57:731-733. Gurney et al. Benefit of vitamin E, riluzole, and gabapentin in a transgenic model of familial amyotrophic lateral sclerosis. Ann. Neurol. 1996, 39: 147-157.
Gurney et al. Motor neuron degeneration in mice that express a human superoxide dismutase mutation. Science 1994, 264: 17721775.
Johnson, K. P. et al. Copolymer 1 reduces relapse rate and improves disability in relapsing-remitting multiple sclerosis: results of a phase III multicenter, double-blind placebo-controlled trial. The Copolymer 1 Multiple Sclerosis Study Group. Neurol., 1995, 45:1268.
Kleywegt, Monoamine oxidase, 1999<http://alpha2.bmc.uu.se/gerard/mao.html>.
Lampert, P. W., Autoimmune and virus-induced demyelinating diseases. A review, 1978, Am. J. Path. 91:17.
Ludolph et al., Antiglutamate therapy in ALS—which is the next step? J. Neural. Transm 1999, 55(Suppl.): 79-96.
McDonald et al., Recommended diagnostic criteria for multiple sclerosis: guidelines from the International Panel on the diagnosis of multiple sclerosis. Ann. Neurol. 2001, 50:121-127.
McKhann, “Multiple Sclerosis” in Encyclopedia of Neuroscience, Birkhauser, Boston, 1987, 705-706.
Rodriguez-Gomez et al., Deprenyl prevents protein oxidation, Brain Res. Mol. Brain. Res., 1997, 46(1-2): 31-38.
Ask the pharmacist: Common questions asked about Rilutek®, ALS Survival Guide, 2002<http://www.lougehrigsdisease.net/drugs/rilutek.html>.
“COPAXONE®” in Physician's Desk Reference, Medical Economics Co., Inc., Montvale, N.J., 2003, 3214-3218.
“Demyelinating Diseases” in Merck Manual of Diagnosis and Therapy, Merck Research Laboratories, Whitehouse Station, N.J., 17th Ed., 1999, 1473-1474.
“Depression” in Merck Manual of Diagnosis and Therapy, Merck Research Laboratories, Whitehouse Station, N.J., 17^{th} Ed., 1999, 1535-1536.
Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. for Biologics Eval. and Res., Clin./Pharm., November 1999<http://www.fda.gov/cber/gdlns/metabol.pdf>.
L-deprenyl, a selective monoamine oxidase type-b inhibitor in endogenous depression, Life Sci., 1980, 26(11): 877-882.
The interaction of L-deprenyl and scopolamine on spatial learning/memory in rats, J. Neural Trans. Parkinson's Dis. Dementia Sect., 1993, 6(3): 189-197.
Monoamine oxidase B inhibitors. Current state and future status, CNS Drugs, 1996, 6(3): 217-236.
Potential applications for monoamine oxidase B inhibitors, Dementia, 1990, 1(6): 323-348.
Rationale for (−)-deprenyl (Selegiline) therapy in Parkinson's disease and Alzheimer's disease, Revista Brasileira de Neurologia, 1995, 31(3): 131-138 (Abstract).
Treatment of Tourette's: An Overview, <http://www.haverford. edu/psych/biopsych217b/tourettes/TSwebtreat.caf.html>.
“RILUTEK®” in Physician's Desk Reference, Medical Economics Co., Inc., Montvale, N.J., 2003, 767-769.
PCT International Publication No. WO 01/52878 (PCT/US01/02117), published Jul. 26, 2001 (Eisenbach-Schwartz et al.).
PCT International Publication No. WO 01/93893 (PCT/US01/02118), published Dec. 13, 2001 (Eisenbach-Schwartz et al.).
Vielhaber S. et al., Mitochondrial DNA abnormalities in skeletal muscle of patients with sporadic amyotrophic lateral sclerosis, Brain, 2000, 123:1339-1348.
Amyotrophic Lateral Sclerosis Information Page, National Institute of Neurological Disorders and Stroke, Jul. 1, 2001<http://www.ninds.nih.gov/health_and_medical/disorders/amyotrophiclateralsclerosis_doc.html>.
PCT Publication No. WO 00/74676, published Dec. 14, 2000 (Vereniging Voor Christelijk Wetenschappelikjk Onderwijs and Biogen, Inc.).
PCT Publication No. WO 01/95907, published Dec. 20, 2001 (Morresearch Applications, Ltd.).
Compston, Genetic susceptibility to multiple sclerosis, in McAlpine's Mutiple Sclerosis, Matthews, B. ed., London: Churchill Livingstone, 1991, 301-319.
Dal Canto, M. C., and H. L. Lipton. 1977. Multiple sclerosis. Animal model: Theiler's virus infection in mice. Am. J. Path. 88:497-500.
Hafler and Weiner, M S: A CNS and systemic autoimmune disease, Immunol. Today, 1989, 10:104-107.
Martyn, The epidemiology of multiple sclerosis, in McAlpine's Multiple Sclerosis, Matthews, B., ed., London: Churchil Livingstone, 1991, 3-40.
Olsson, Immunology of multiple sclerosis, Curr. Opin. Neurol. Neurosurg., 1992, 5:195-202.
Rodriguez, M. et al. 1987. Theiler's murine encephalomyelitis: a model of demyelination and persistence of virus. Crit. Rev. Immunol. 7:325.
Multiple sclerosis: its diagnosis, symptoms, types and stages, 2003<http://www.albany.net/˜tjc/multiple-sclerosis.html>.

1. A method of providing neuroprotection to the central or peripheral nervous system of a subject in need of such neuroprotection comprising periodically administering to the subject an amount of glatiramer acetate and an amount of 2-amino-6-trifluoromethoxybenzathiazole, wherein the amounts when taken together are effective to provide neuroprotection to the central or peripheral nervous system of the subject.

2. The method of claim 1, wherein providing neuroprotection comprises treating a neurodegenerative disease.

3. The method of claim 2, wherein the neurodegenerative disease is multiple sclerosis, amyotrophic lateral sclerosis, acute disseminated encephalomyelitis, adrenoleukodystrophy, adrenomyeloneuropathy, Leber's hereditary optic atrophy, Human Lymphotrophic T-cell Virus I (HTLVI)-associated myelopathy, acute viral encephalitis, aseptic meningitis, virus-induced demyelination, demyelinating genetic diseases, transverse myelitis, Progressive Multifocal Leucoencephalopathy, a nutritional metabolic disorder, acute glaucoma, chronic glaucoma, close-angle glaucoma, open-angle glaucoma, optic neuritis or systemic lupus erythematosus.

4. The method of claim 3, wherein the neurodegenerative disease is multiple sclerosis.

5. The method of claim 3, wherein the neurodegenerative disease is amyotrophic lateral sclerosis.

6-8. (canceled)

9. The method of claim 1, wherein providing neuroprotection comprises treating neurotrauma.

10. The method of claim 9, wherein the neurotrauma is a result of a traumatic event selected from the group consisting of head trauma, spinal trauma, neurotoxic injury, eye injury, stroke, ischemia, hypoxia, and anoxia.

11. (canceled)

12. The method of claim 1, wherein providing neuroprotection comprises providing protection against toxic levels of glutamate.

13. The method of claim 1, wherein providing neuroprotection comprises providing protection against toxic levels of monoamine oxidase-B activity.

14. The method of claim 1, wherein the subject is a human being.

15. The method of claim 1, wherein each of the amount of glatiramer acetate when taken alone, and the amount of 2-amino-6-trifluoromethoxybenzathiazole when taken alone is effective to provide neuroprotection to the central or peripheral nervous system of the subject.

16. The method of claim 1, wherein either the amount of glatiramer acetate when taken alone, the amount of 2-amino-6-trifluoromethoxybenzathiazole when taken alone or each such amount when taken alone is not effective to provide neuroprotection to the central or peripheral nervous system of the subject.

17. The method of claim 1, wherein the amount of glatiramer acetate is in the range from 10 to 600 mg/week.

18-21. (canceled)

22. The method of claim 1, wherein the amount of glatiramer acetate is 20 mg/day.

23-26. (canceled)

27. The method of claim 1, wherein the administration of the glatiramer acetate substantially precedes the administration of the 2-amino-6-trifluoromethoxybenzathiazole.

28. The method of claim 1, wherein the administration of the 2-amino-6-trifluoromethoxybenzathiazole substantially precedes the administration of the glatiramer acetate.

29. The method of claim 1, wherein the administration of the glatiramer acetate is effected subcutaneously, intraperitoneally, intravenously, intramuscularly, intraocularly or orally and the administration of the 2-amino-6-trifluoromethoxybenzathiazole is effected orally.

30. The method of claim 29, wherein the administration of the glatiramer acetate is effected subcutaneously and the administration of the 2-amino-6-trifluoromethoxybenzathiazole is effected orally.

31-45. (canceled)

46. A pharmaceutical composition comprising an amount of glatiramer acetate and an amount of 2-amino-6-trifluoromethoxybenzothiazole, wherein the amounts when taken together are effective to provide neuroprotection to the central or peripheral nervous system of a subject in need of such neuroprotection.

47. The pharmaceutical composition of claim 46, wherein each of the amount of glatiramer acetate when taken alone and the amount of 2-amino-6-trifluoromethoxybenzathiazole when taken alone is effective to provide neuroprotection to the central or peripheral nervous system of the subject.

48. The pharmaceutical composition of claim 46, wherein either of the amount of glatiramer acetate when taken alone, or the amount of 2-amino-6-trifluoromethoxybenzathiazole when taken alone or each such amount when taken alone is not effective to provide neuroprotection to the central or peripheral nervous system of the subject.

49-110. (canceled)
